(19) United States
(12) Patent Application Publication
(10) Pub. No.: US 2015/0105608 A1
(43) Pub. Date: Apr. 16, 2015

(54) Infant Sleeping Aid and Infant-Bed Accessory

(71) Applicant: Rest Devices, Inc., Boston, MA (US)

(72) Inventors: Thomas Lipoma, Boston, MA (US); Katy Gero, Cambridge, MA (US)

(21) Appl. No.: 14/513,649

(22) Filed: Oct. 14, 2014

Related U.S. Application Data
(60) Provisional application No. 61/890,380, filed on Oct. 14, 2013.

(51) Int. Cl. A61M 21/02; A63H 33/00; A61B 5/00; A61B 5/11; A61B 5/01

(52) U.S. Cl. CPC A61M 21/02; A61B 5/1118; A61B 5/01; A61B 5/4809; A63H 33/006; A61M 2021/0027
USPC 600/27

(57) ABSTRACT

A method and system for controlling an infant-bed accessory is disclosed. The system employs collected information associated with the infant or the caregiver to initiate controls of the infant-bed accessory without the user's interaction.

### PRIORITY

The present U.S. patent application claims priority from U.S. Provisional patent application Ser. No. 61/890,380 filed on Oct. 14, 2013, entitled “Infant Sleeping Aid and Infant Crib Accessory, the contents of which are incorporated herein in their entirety.

### TECHNICAL FIELD

The present invention relates to an infant sleeping aid, and more particularly, to controlling an infant-bed accessory that aids an infant to sleep.

### BACKGROUND

Parents and caregivers often place calming devices in nurseries to help lull an infant to sleep when the infant is falling asleep or attempting to fall asleep. Such calming devices may be a moving mobile, which may also produce soothing sounds or calming lights. Such mobile may be attached to or place near the crib.

Existing crib mobiles may use a combination of lights, sounds, and motion. However, use of these devices is typically initiated by the caregiver themselves.

### SUMMARY

In accordance with a first embodiment of the invention, there is provided a method of controlling an infant mobile. In this embodiment, the method includes connecting the infant mobile to a network, such as a home area network, or a cellular network. Alternatively, the infant mobile may connect by a point-to-point connectivity with a personal computing device. The method includes transmitting information between a personal computing device and the infant mobile through the network. The system may then change the state of the infant mobile based on the transmitted information. The infant mobile may include a set of lamps or light-emitting diodes to output lights, a speaker to output sound, and a motor to rotate the rotary member. The light outputs may be configurable in intensity and color. The infant mobile may include an electronic relay for powering an electrical outlet.

The infant mobile may sense movements associated with the infant and transmit the sensed movement to a personal computing device. The sensed movement is preferably sensed with an infrared motion detector.

The system may use collected information about the infant mobile, the infant, the user (such as the caregiver), or the environment associated with the infant. The collected information is used to determine when to initiate control states of the system or an effective output state of the infant mobile.

Information about the user may include the user's identity, the user's location, and the user's proximity from the device. The system may collect or derive usage statistics associated with the usage of the infant mobile for each infant. Such information may include time of use information and the user identity. Information about the infant may include the infant age, weight, time asleep, time awake, temperature, movement, mood, and/or sound levels. Environmental information may include room temperature, location, light levels, sound levels, time, humidity levels, and/or proximity of low flying aircraft.

In another related embodiment, the information may be collected and stored within the system's memory over time for analysis to create a usage schedule of the infant. The schedule may be displayed or utilized to alert the user of upcoming sleeping events. The system may determine if a user is likely to change the state of the mobile. In particular, determining if a user is likely to change the state of the infant mobile is preferably based on a determining if the infant is awake. This determination may be a received command from the network, including the internet, a personal computing device over the internet, or wirelessly to a personal computing device. In another embodiment, the determination may be based on a determining of a mood of the infant. In various embodiments, the changing of the state of the infant mobile is performed without an interaction of the user.

In accordance with another embodiment, the system may determine a likelihood value associated with the activation of the infant mobile based on the received state information. The system may cause the initiation of the control state based on the likelihood value exceeding a specified threshold. The state information may be selected from a group consisting of a subject's awake state, a subject sleep state, a subject's quiet asleep state, a subject's active sleep state, a subject's drowsy awake state, a subject's quiet alert awake state, subject's quiet state, a subject's deep sleep state, a subject's light sleep state, a subject's alert state, a subject's active state, a subject's active alert state, a crying cue, a fussing cue, a spitting cue, a gagging cue, a jittery cue, a jerky cue, a frowning cue, a red cue, a pale cue, an agitated cue, a thrashing cue, a falling sleep cue, an averting gaze cue, a smiling cue, a smooth motor movement cue, and a subject orientation. The state information may be collected from sensors selected from a group consisting of a microphone, a temperature sensor, an accelerator, a capacitance sensor, an inductance sensor, a gyroscopic sensor, a scale, a conductance sensor, and an infrared sensor. The state information may be determined from measured movements of the subject or collected from an external infant monitoring apparatus.

In accordance with another embodiment, the system may determine the proximity of an electronic device to the infant mobile. The system may then initiate the control states based on the proximity. The proximity may be determined based on a presence of the electronic device being within a distance to the infant mobile. The system may receive state information associated with the infant and cause the initiation of the control state based on the state information and the proximity. The initiation of the control state may be based on the proximity of the electronic device being within a specified distance and/or a specified time, which may be an input received from the electronic device. An electronic device may be a personal computing device selected from a group consisting of: mobile phones, cellphones, smartphones, tablets, mobile computing devices, laptops, computers, desktops, servers, and radio transmitters.

In accordance with another embodiment, the system may determine a mood state of the infant based on collected information associated with the infant. To this end, the system may cause the initiation of a control state of infant mobile based on the mood state.

In accordance with another embodiment, an infant-bed accessory is described. The system may include a sensory-stimulus member configured to be placed in proximity to a subject's bed. The system include a sensor configured for sensing an attribute of the subject and a controller configured for receiving signals from the sensor. The controller is configured to determine a wake state and/or a sleep state of the subject based on the received signals. The controller determines a recommended sleep environment for the subject based on the determined state and causes a report of the recommended sleep environment to be sent to an external user portal. The external user portal operates on an at least one electronic communication device selected from a group consisting of a cell phone, a smart phone, a tablet, and a personal computer.

The recommended sleep environment may include: a future time to put the subject to sleep, a value associated with humidity levels near the subject's bed, a value associated with temperature levels near the subject's bed, a value associated with light levels near the subject's bed, and a value associated with sound levels near the subject's bed.

In accordance with another embodiment, the infant-bed accessory includes the sensory-stimulus member configured to be placed above the subject's bed. The sensory-stimulus member having at least one configurable electrically-actuated output selected from a group consisting of lights, sounds, and motion. A controller is operatively connected to the sensory-stimulus member to control the at least one configurable electrically-actuated output. The controller is configured to (i) receive signals associated with an awake state or a sleep state of the subject and to (ii) cause a sequence of the configurable electrically-actuated output that has a determined highest likelihood of lulling to the subject to sleep based on the received signals.

In an embodiment, the sequence may be received as a parameter from an external device. In another embodiment, the controller may determine the sequence. The controller may maintain a record, in memory, of a plurality of values associated with a running-configuration of the configurable electrically-actuated output. The controller may then update the plurality of values based on a time for the subject to fall asleep while the running-configuration is initiated. The record may be maintained as a histogram.

In accordance with another embodiment, the infant-bed accessory may include a sensory-stimulus member configured to be placed above the subject's bed. The sensory-stimulus member may have at least one configurable electrically-actuated output selected from a group consisting of lights, sounds, and motion. The sensory-stimulus member may include a plurality of lights. The system may include a sensor configured for sensing an attribute of the subject and a controller configured for receiving signals from the sensor. The controller may be configured, upon the sensory-stimulus member being initiated to output, to determine a wake state and/or a sleep state of the subject based on the received signals. The controller may then cause an output of a report to an external user portal upon the determined state being associated with the subject being asleep.

In the various embodiments, the system may include an attachment member for mounting to a crib or a body for supporting the accessory in an upright position and for housing the controller.

In accordance with another embodiment, a method of controlling an infant-bed accessory is disclosed. The method may include receiving a first sensor input associated with a presence of a subject in a subject's bed and a second sensor input associated with an awake state of the subject in the subject's bed. The method may include determining, via a processor, whether the subject is present in the subject's bed based on the first sensor input. The method may determine, via the processor, whether the subject is awake if the subject is determined to be present and cause an initiation of a mobile if the subject is determined to be awake.

The first sensor may include at least one type of sensor selected from a group consisting of a motion sensor, a camera, an infrared sensor, and capacitance sensor, and an inductance sensor. The second sensor may be a type selected from a group consisting of an infrared sensor, a capacitance sensor, a temperature sensor, a light sensor, a chemical sensor, motion sensor, a humidity sensor, a vibration sensor, a pressure sensor, an electric field sensor, a sound sensor, a stretch sensor, and a biosensor. The sensor may be configured to sense at least one physical property of the subject selected from a group consisting of: the subject's heart beating rate, the subject's oxygen saturation level, the subject's movement, the subject's location, the subject's noise level, the subject's body temperature, the subject's skin conductivity; the subject's skin moisture level, and the subject's infrared light emission.

The controller may include a memory configured to store a control sequence for the plurality of lights, the sequence including at least one parameter selected from a group of parameters associated with a light's color, a light's intensity, and a light's pulsing frequency. The sensory-stimulus member includes an electronic speaker operatively connected to and controlled by the controller, and wherein the controller includes a memory for storing at least one audio sequence to output to the electronic speaker. The sensory-stimulus member may be configured to rotate, or to emit light, or to emit sound when initiated. The controller may be configured to receive an audio file over a communication network. The controller may store the received audio file in the memory as a stored audio sequence to output to the electronic speaker.

The system may include a switch member for gating power to an AC socket. The switch member may be operatively connected to the controller configured to monitor power through the switch member.

### BRIEF DESCRIPTION OF THE DRAWINGS

The foregoing features of embodiments will be more readily understood by reference to the following detailed description, taken with reference to the accompanying drawings, in which:

FIG. 1 schematically illustrates an infant sleeping aid and bed accessory system according to an illustrative embodiment of the invention;

FIG. 2 illustrates a detail schematic of the infant accessory system of FIG. 1 according to an embodiment of the invention;

FIG. 3 schematically illustrates the infant accessory system coupled to a controller for sensing physical attributes of infants according to an embodiment of the invention;

FIG. 4 schematically illustrates a method of operation of the infant-bed accessory system according to an embodiment of the invention;

FIG. 5 schematically illustrates another method of operation of the infant-bed accessory system according to an embodiment of the invention;

FIG. 6 schematically illustrates another method of operation of the infant accessory system according to an embodiment of the invention;

FIG. 7 schematically illustrates a method of determining a likelihood value for operating the infant-bed accessory system according to an embodiment of the invention;

FIG. 8 schematically illustrates a method of controlling the output of the electrically-actuated outputs according to an embodiment of the invention;

FIG. 9 schematically illustrates another method of operation of the infant accessory system according to an embodiment of the invention; and

FIGS. 10-12 show exemplary display screens that may, for example, be displayed on a personal computing device in accordance with various embodiments of the invention.

### DETAILED DESCRIPTION OF SPECIFIC EMBODIMENTS

As used in this description and the accompanying claims, the following terms shall have the meanings indicated, unless the context otherwise requires:

The term “infant-bed” generally refers a sleeping area of an infant, such as a crib and beds with bars or latticed sides.

The term “infant” refers to very young children less than five years of age. The term is interchangeably used with the term “subject.”

In illustrative embodiments of the invention, a novel control system and methodology for an infant sleeping aid and bed accessory, such as an infant mobile, is disclosed. The system and method employ information associated with both the infant and the infant's caregiver to assist the caregiver in the care of the infant, particularly in getting the infant to sleep and keeping the infant asleep. Details are described below.

In various embodiments of the invention, the system is placed in proximity to an infant's bed to provide a sensory stimulus, such as lights, sounds, and/or motion, to lull an infant to sleep. The system begins using preferences provided by the user to provide the sensory stimuli to the infant. The term “user” is interchangeably used herein with the term “caregiver.” Over time, the system identifies environmental conditions as well as sensor stimuli in which the infant is most responsive. These stimuli may include sequences of configurable electrically-actuated outputs of the system. Consequently, after providing the initial preferences, the system can operate with little or no intervention from the user (for example, the caregiver).

The caregiver may set the level of information he or she would like to receive from the system. For example, the system may generate and provide alerts as to specific sleeping events, as well as summary reports, and recommendations to the caregiver. The recommendations may include the conditions and stimuli identified to be the most effective and may be customized for specific goals of the caregiver, particularly in the sleep training of the infant.

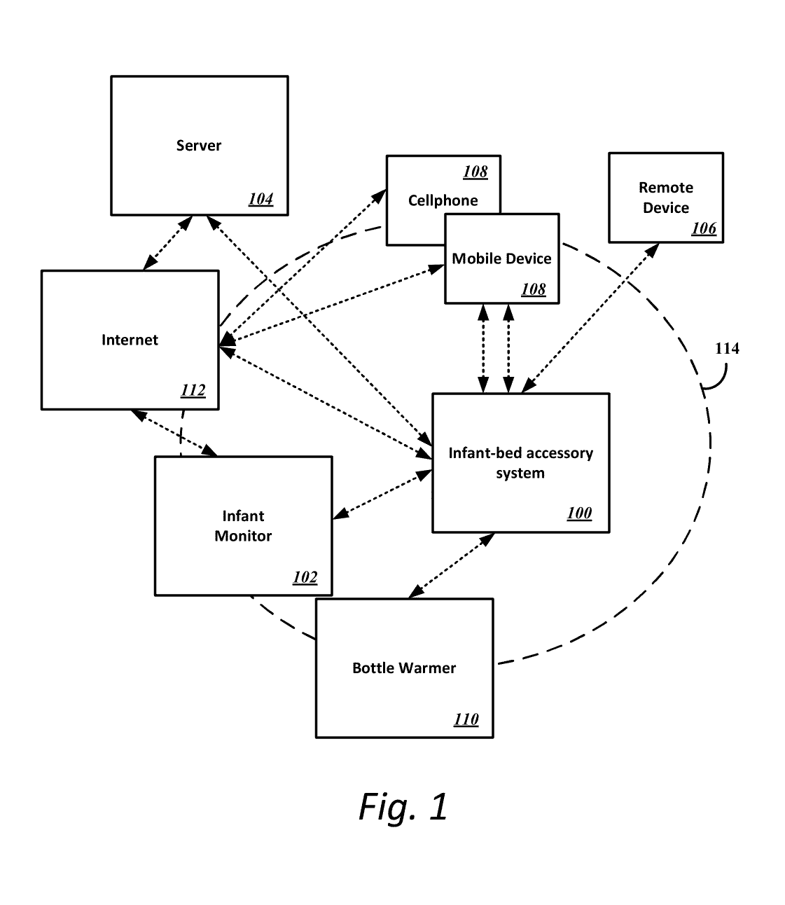

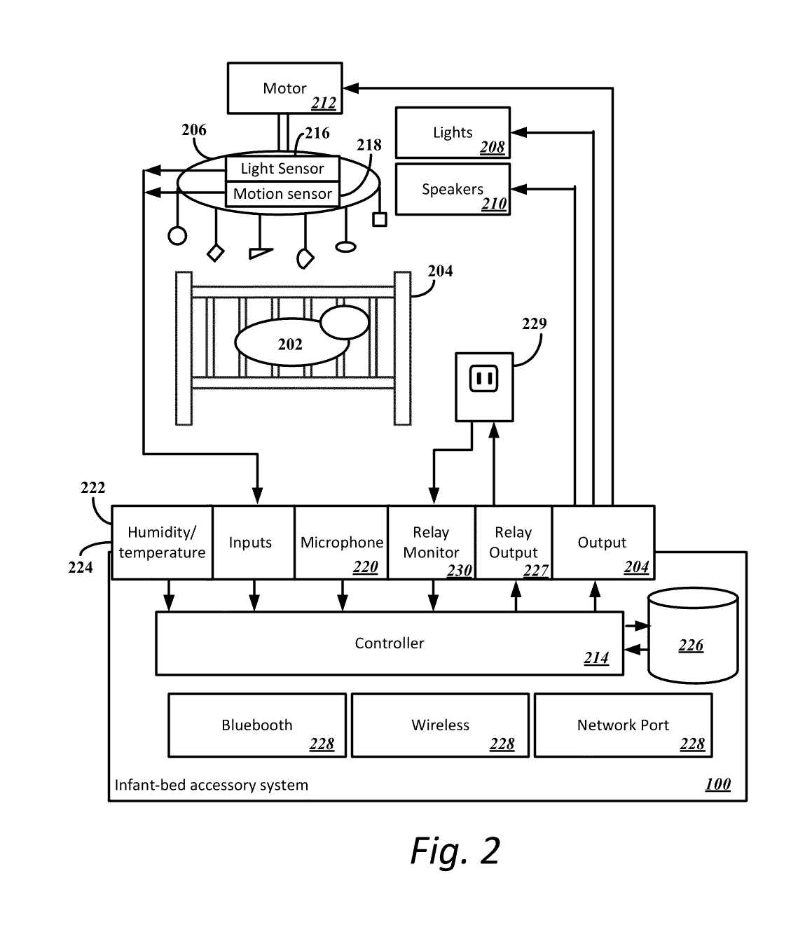

FIG. 1 schematically illustrates an infant sleeping aid and bed accessory system 100 according to an illustrative embodiment of the invention. The system 100 (referred to in the figure as “infant-accessory system 100”) is configured to provide calming stimuli to an infant 202 (see FIG. 2) in order to lull the infant to sleep, or back to sleep. Preferably, the system 100 includes a rotating member, such as an infant/toddler mobile, that slowly rotates (for example, slower than one revolution per second). Additionally, the system may include lights or a speaker to provide calming lights and sounds. The lights, sound, and motion from the system are examples of electrically-actuated outputs and may be configurable to provide a wide range of combinations. The system may determine the most effective electrically-actuated outputs for each infant using observed responses of the infant. The number of combinations of electrically-actuated outputs is preferably greater than 100, even more preferably between 100 and 2,000 combinations.

The system 100 preferably includes at least one communication port to operatively communicate with at least one external device, such as an infant monitor 102, a server 104, a remote device 106, a communication device 108 (for example, a mobile phone or a mobile computing device), or a bottle-warmer 110. The apparatus 100 may communicate directly with such devices or indirectly over a network 112 (for example, Internet, wide area network, and/or local area network). The communication port provides means for the system 100 to provide notification and alerts to the caregiver, to communicate with an external data processing system providing analytics to improve care of the infant, and to coordinate operations with other care function (for example, health monitoring and/or infant-feeding system).

FIG. 2 illustrates a detail schematic of the infant accessory system 100 of FIG. 1 according to an embodiment of the invention. The system 100, or a portion thereof, is configured to be placed preferably above an infant bed 204. The system 100 may include a rotary member 206, which may be ornate as an infant mobile. The system 100 may include lights 208 and a speaker 210 to produce lights and sound. The lights 208 and speaker 210 are preferably disposed at the rotary member 206. In an embodiment, the lights 208 may be disposed along a perimeter of the rotary member 206, for example, in a ring. The rotary member 206 is preferably coupled to a motor 212 for slowly rotating the rotary member 206.

The system 100 includes a controller 214 that may record and/or maintain, without limitation, information associated with the environment around system 100. In being placed near and above the infant bed 204, the system 100 records information associated with the infant and the environment surrounding the infant's bed. The controller 214 may also record usage information associated with the configurable electrically-actuated outputs of the system 100 (for example, the lights 208, the speaker 210, and the motor 212) as well as various information associated with the infant received from external devices 102, 106, 108, 110 or the networks 104, 112. Specifically, the information may include:
Time of use (for example, time and date and/or frequency) of the system 100;
Configuration of the electrically-actuated outputs of the system 100 during use (for example, light output configurations, including color output, brightness output, and output intermittency; sounds output, including titles of songs, noise, or lullabies, and sound level; and motor output, including on/off state and rotation speed);
Environment condition (for example, room temperature, room light levels, room humidity levels, room ambient sound levels);
Presence of caregiver (for example, the proximity and/or identity of people in the same room as the infant; proximity or presence of a communication device 108 associated with the caregiver);
Infant's physiological state (for example, movement, heart rate, breathing rate, body temperature, skin temperature, blood oxygen saturation level, location);
Infant information (for example, age, weight, time asleep, time awake, body temperature, movement, mood, and sound levels); and
Controls information (for example, total run-time over a defined time) of the system 100.

In the embodiment shown, the system 100 may include a light sensor 216, a motion sensor 218, a microphone 220, a temperature sensor 222, a humidity sensor 224, and various other sensors for collecting the recorded information, described above. Some of the sensors, such as the light sensor 216 and motion sensor 218, may be disposed at the rotary member 206. The recorded information may be stored in memory 226. Other sensor that may be used includes, but not limited to, an infrared sensor, a capacitance sensor, a chemical sensor, a vibration sensor, a pressure sensor, an electric field sensor, a stretch sensor, and a biosensor. Of course, the sensors may be employed in various configurations, such as in a stationary base station of the system 100.

The system 100 may be configured with an attachment member to fixably mount to the railing of an infant bed 204 or a base to stand next to the infant bed 204. In the later, the system 100 may include a base portion, a shaft portion, and a hanging portion that hangs over the infant bed 204. Of course, other form factor may be employed. For example, the system 100 may configured as a tabletop device or as a hanging device configured to attach to the ceiling.

The system 100 preferably includes communication ports 228 for interfacing to a network to receive signals and data to control its operation, including the initiation of various infant-bed accessory and network controls. The communication port 228 may communicate, for example, via a Bluetooth transceiver (for example, IEEE 802.15.1), a Wi-Fi transceiver (for example, IEEE 802.11), a Zigbee transceiver (for example, IEEE 802.15), or a FM/AM radio transceiver to receive control signals from the wireless network or directly from the infant monitor 102, the server 104, the remote device 106, the communication device 108, or the bottle-warmer 110. Other wireless communication transmissions and protocols may be employed, particularly those that allow for communication across multiple rooms. Communication device 108 may include a personal computing device, including, without limitation, a personal phone, a computer, a tablet, or any other suitable electronic device which is connected to the internet and can display an output. The system 100 may communicate its collected information (for example, sensor and usage information) to the server 104 via the network for storage and analysis over time.

The connectivity allows for the caregiver to remotely interact with the infant. To this end, the caregiver may employ the various features to train the infant, particularly to sleep for long continuous period of time, and to coup with the caregiver not being nearby when asleep or falling asleep.

Still looking at FIG. 2, the system 100 may include a relayed power output 227 to provide controllable power to a standard wall socket 229. The relayed power output 227 may include a monitoring circuit 230 to monitor usages of the wall socket 229. To this end, the system 200 may monitor the wall socket 229 to determine usage patterns of an external device connected thereto (such as a lamp). In an embodiment, the controller 214 may determine whether a correlation exists between the usages of the wall socket 229 and either the sleep pattern of the infant or caregiver usage pattern of the system 100. The controller 214 may then initiate the controls of the relay output 227 based on a determined correlation of the usage.

The system 100 may utilize proximity information 114 (see FIG. 1) of the caregiver as a trigger or condition for a control action. The proximity information 114 may be based on spatial information (for example, GPS data or mobile location information) or wireless network information (for example, a presence of a wireless device or the signal strength of the device or the presence of a broadcast message). The proximity information 114 may also be based on sensor outputs, such as those of the motion sensor 218.

In an embodiment, the system 100 may use proximity, for example, to enable a lamp placed near the infant-bed and connected to the wall socket 229. Here, the system 100 has determined a correlation between the infant waking up and the caregiver entering the room and turning on the lamp. As such, once the system 100 determines this correlation, the system 100 may use the proximity of the caregiver to initiate the turning-on of the lamp so the caregiver would not have to. The system 100 may be configured to request permission from the user when a new pattern or habit is identified.

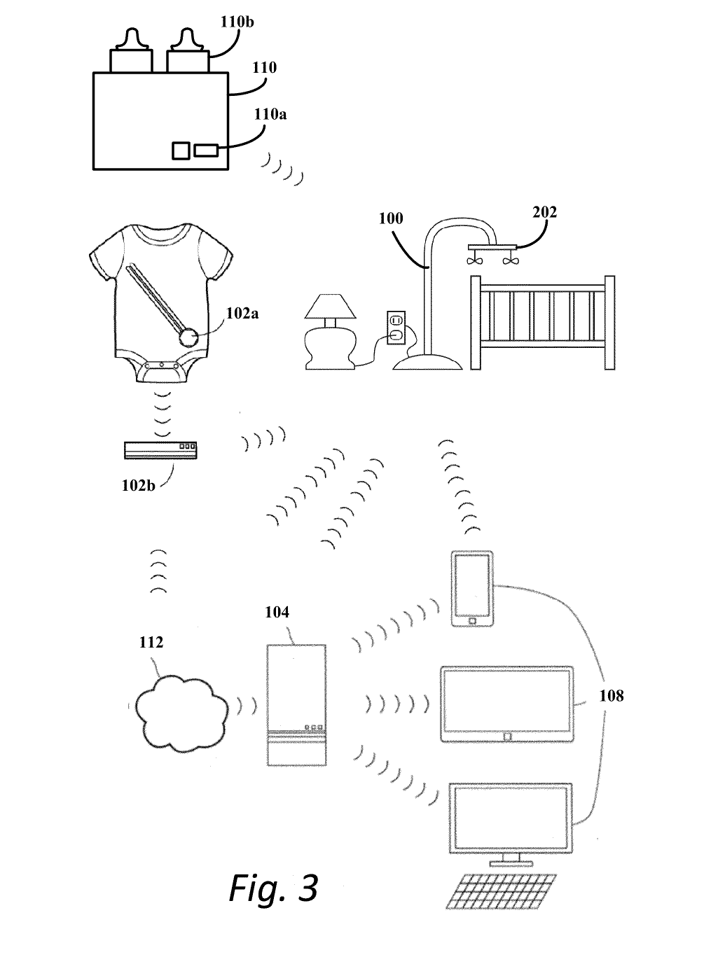

FIG. 3 schematically illustrates the infant-bed accessory system 100 coupled to an infant monitor 102, a bottle warmer 110, or other external and intermediate devices (104, 108, 112) for monitoring sleep of the infant according to an embodiment. The infant monitor 102 may include a controller 102a for monitoring information from sensors embedded in articles of clothing of the infant. The controller 102a may interface directly to the system 100 or through an intermediate base station 102b. The base station 102b and the bottle warmer 110 may be alternatively configured to transmit the collected information either to the server 104, which then stores and analyze the information to provide commands to the system 100, or directly to the system 100. The bottle warmer 110 may include a controller 110a for monitoring the feeding of the infant by monitoring the usage of the bottles 110b.

Examples of the controller of the article of clothing are described in U.S. patent application Ser. No. 13/745,098, and U.S. provisional application, Ser. No. 61/713,177, by Lipoma et al. An example of bottle warmer is described in U.S. patent application, Ser. No. 61/875,310, by Lipoma et al. These applications are incorporated by reference herein in their entireties.

It should be appreciated by those skilled in the art that the data of the infant monitor 102 may be used independent of the bottle warmer 110, and the bottle warmer 110 may be used independent of the infant monitor 102. Though, either one of the systems 102, 110 or both systems 102, 110 may be used in conjunction with the present system 100 to improve the monitoring of the sleep of the infant.

For example, the infant monitor 102 and the bottle warmer 110 may collect information associated with sleep of the infant to augment the sensor reading of the present system 100. These additional sensor readings may be associated with the various states of the infant, the physiological responses of the infant, movements of the infant, environmental condition around the infant, and the orientation of the infant.

State information may include, for example, mental or body or behavioral state information relating to:
The infant's awake state (for example, alert awake state, drowsy awake state, quiet alert awake state);
The infant's sleep state (for example, quiet sleep state, active sleep state, deep sleep state, light sleep state);
The infant's hunger state; and
The infant's cues (for example, crying cue, fussing cue, spitting cue, gagging cue, jittery cue, jerky cue, frowning cue, red cue, pale cue, agitated cue, thrashing cue, falling sleep cue, averting gaze cue, smiling cue, smooth motor movement cue).

Environmental condition around the infant may include sound, room temperature levels, humidity levels, light levels, and the presence of people in the room.

Physiological responses may include information associated with body functions of the infant, including heart rate, blood oxygen saturation level, breathing rate, perspiration rate, body temperature, skin temperature, voluntary muscle movement and involuntary muscle movement.

The collected information allows the system 100 to determine whether the infant is awake or about to wake as well as whether the infant is hungry or merely irritated. It should be appreciated by one skilled in the art that determining the infant awake state may also be determined based on the infant's sleep state. The system 100 may use either awake or asleep information from the infant monitor 102. The determined likelihood information may be further combined with information associated with the caregiver to improve the reliability of the prediction, particularly in determining the likelihood to initiate the electrically-actuated outputs (to lull the infant to sleep).

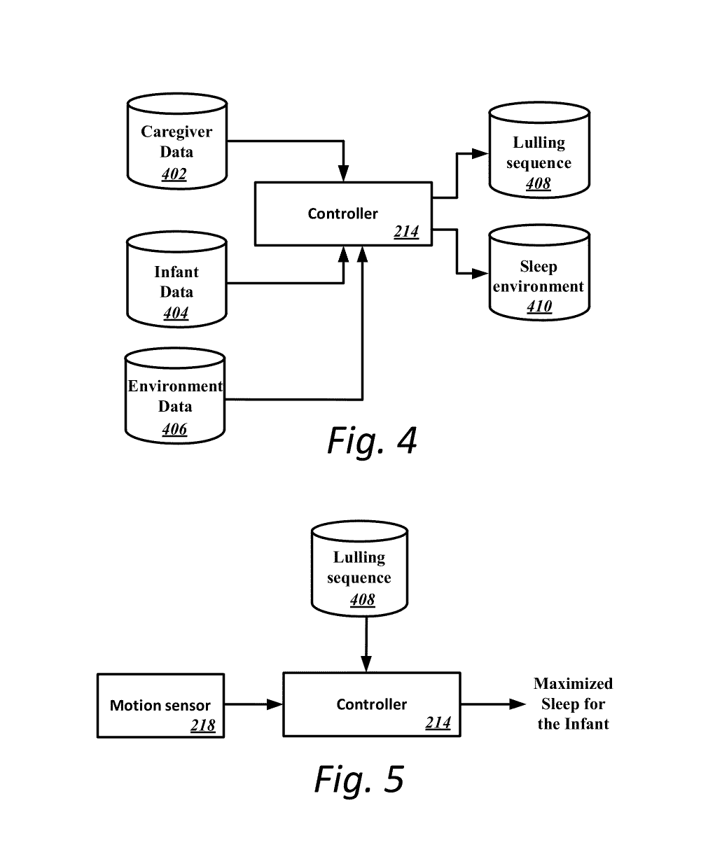

FIG. 4 schematically illustrates a method of operation of the infant-bed accessory system 100 according to an embodiment. The system 100 monitors and learns both the actions of the caregiver and response of the infant 202. The information associated with the caregiver may be stored as caregiver data 402 and may include
When the caregiver has modified settings of the system 100;
Configuration settings that has been modified by the caregiver; and
When the caregiver has initiated the electrically-actuated outputs.

The information associated with the infant may be stored as infant data 404 and may include:
When the infant 202 fell asleep;
Time for the infant 202 to fall asleep under a configuration setting of the electrically-actuated outputs (208, 210, 212);
Awake time and/or frequency;
Sleep time and/or frequency;
Movements;
Skin temperature;
Crying time and/or frequency;
Mood; and
Hunger state.

The information associated with environment condition may be stored as environment data 406 and may include:
Room temperature;
Room light levels;
Room humidity levels; and
Room ambient sound levels.

In an aspect of the embodiment of the invention, the controller 214 employs the infant data 404 to determine a recommended setting for the electrically-actuated outputs (208, 210, 212). The recommended setting is stored in memory 226 and is referred to in the figure as a “lulling sequence 408.” The system 100 may utilize the caregiver data 402 to ensure that the lulling sequence 408 is consistent with preferences of that of the caregiver. For example, if the caregiver sets a lullaby, sound file, or music type or a certain light output setting (for example, blue over green lights), the system 100 may weigh such configurations as preferences over configurations not selected by the caregiver.

In an embodiment, the controller 214 may maintain the data, as collected over time, in a histogram. Of course, the data may be maintained in other data structure, such as, but not limited to, hash tables. The histogram may be configured as a set or partial set of all combinations of the electrically-actuated outputs (208, 210, 212).

The system 100 includes preferably between 25 and 50 light output settings, preferably at least 10 sound output settings, and preferably at least 2 motion settings. As such, a system 100 with 50 light output settings, 10 sound output setting, and 2 motion setting has 1,000 output combinations.

Light output settings may include preferably between 5-10 light output colors, preferably 3 brightness level, and preferably an option for pulsing the outputs (i.e., on and off). An example of light output colors may include red, orange, pink, green, blue, violet, white, yellow, and purple. The brightness level may be low, medium, and high. The intermittent output may be at a frequency consistent with a desired calm breathing rate of the infant 202, which may vary preferably between 0.1 and 1 Hz. Of course, other colors, brightness levels, and output frequency may be employed as well as customizable by the user. The pulse output may be ramped, such as by fading in and out, to provide a continuous and smooth output, also at a frequency consistent with the desired calmed breathing rate of the infant 202. Sound output settings may include pink noise, white noise, water noise, and various lullabies, tunes, and songs. Custom song may be received from external devices and networks through the communication ports 228 during usage or stored in the memory 226 for later usage. In particular, the controller 214 may record speech received from a personal communicating device 108 and store it as an audio file that may be used as a customized sound output. To this end, the voice of the caregiver or user may be stored as a lullaby or a message to be played to the infant 202 as an electrically-actuated output.

Motion setting is preferably one and off and is preferably kept constant at a rate preferably between 0.1 and 1 Hz. The pulse output, motor speed, and sound tempo may be varied to be at a rate consistent with the desired calm breathing rate of the infant. For example, for a newborn to a 6-month old infant, the rate may be between 30-60 pulses per minute; for 6 to 12-month old infant, 24-30 pulses per minute; and for 1-5 year old infant, 20-30 pulses per minute.

The controller 214 may maintain a first histogram for the preferences of the caregiver and a second histogram for the reaction of the infant to the selected lulling sequence of the electrically-actuated outputs. The controller 214 may determine an effectiveness score of each combination in the histogram. An example the effectiveness score calculation is in Equation 1.

$$Effectiveness Score ( x ) = a \frac{p ( x )}{t} + b \frac{1}{s ( x )} \qquad ( Equation 1 )$$

where x is the setting number, a is a preference constant, p(x) is the number of times the caregiver has selected the setting, t is the total number of times the caregiver has selected any settings, b is an effectiveness constant, and s(x) is average time for the infant to fall sleep with the setting. In using the number of times a caregiver has selected a setting, other settings are not penalized due to a system 100 being left in the setting for a period of time. The controller 214 may select and store the combination, x, having the highest effectiveness score as the lull sequence 408. In the event several combinations have the same highest effectiveness scores, the controller 214 may randomly select among such combinations.

The controller 214 may evaluate the combinations in stages to improve the learning time. In an embodiment, the controller 214 may first evaluate the effectiveness of each type of the electrically-actuated outputs (208, 210, 212). For example, the controller 214 may determine whether the infant 202 reacts to light, sound, or motion. As such, non-effective output types may be determined and discarded from further evaluation. The system may then evaluate settings that cause a desired response from the infant. In an embodiment, the evaluation may be based on a decision tree methodology.

In another aspect of the embodiment, the controller 214 may experiment with the setting outputs to refine the lulling sequence 408. The experimentation sequence maybe employed to acquire information about specific combinations of the electrically-actuated outputs (or corresponding portions of the histogram) that has no or small amount of information. To this end, the experimentation sequence may cease after the specific combinations of the electrically-actuated outputs, or the histogram, has a sufficient number of data points above a specified threshold.

The controller 214 may experiment with types of output settings based on the proximity or presence of the caregiver. In an embodiment, when the caregiver is not in the room (such as in the middle of the night), the controller 214 may experiment with settings not previously selected by the caregiver. For example, the controller 214 may employ different colors, or pulsing output. In contrast, the controller 214 may experiment with slight variations of the output settings when the caregiver is nearby. The slight variations may include varying the brightness intensity levels of the selected color output or varying the color output only to adjacent colors (for example, from red to pink or orange). The controller 214 may also employ known likes or dislikes of the infant and avoid output settings having a low likelihood of success.

In another aspect of the embodiment of the invention, the controller 214 employs the caregiver data 402 and infant data 404 to determine a recommended sleep environment for the infant 202. The recommendations are stored in memory 226 and are referred to in the figure as “sleep environment 410.” The sleep environment 410 may include:
Time when the infant is most likely to be sleepy;
Time when the infant is likely to sleep for the longest period within a 24-hour period;
Time when the infant is likely to sleep for the longest period during the evening;
Time when the infant is likely to sleep for the longest period without requiring attention of the caregiver;
Room temperature levels; and
Room humidity levels.

A subset of the recommended sleep environment 408 that are associated only with time is referred to as the recommended sleep time 408a.

The controller 214 may notify the caregiver of detected uncomfortable environment conditions, such as, for example, high skin temperature of the infant 202.

Various regression, clustering, or modeling analysis may be employed to detect patterns in the infant data 404 and the environment data 406.

FIG. 5 schematically illustrates another method of operation of the infant-bed accessory system according to an embodiment. The system 100 employs sensor readings associated with the infant, shown as motion sensor 218, to determine a wake state or a sleep state of the infant. The system 100 then employs the lulling sequence 408 and the recommended sleep time 410a to maximize the sleep of the infant and/or minimize the interaction of the caregiver.

In an embodiment, the system may initiate the electrically-actuated outputs based on (i) a manual input from the user, (ii) a schedule provided by the user, and (iii) a determination, if given permission by the user, based on analyzed infant behavior. Upon the system 100 being initiated to lull the infant, the controller 214 then begins determining whether the infant is asleep based on sensor readings from the motion sensor 218. The determination of the sleep and/or wake state of the infant may be based on the change in rate of the movements of the infant exceeding a pre-determined threshold. Upon the controller 214 determining that the infant 202 is asleep, the controller 214 may cease the operation of the electrically-actuated outputs and cause an output of a report to the caregiver indicating that the infant 202 is asleep. In the event that the infant does not fall asleep within a specified time, the usage is recorded as ineffective. When the infant wakes, the system 100 may initiate operations of the electrically-actuated outputs.

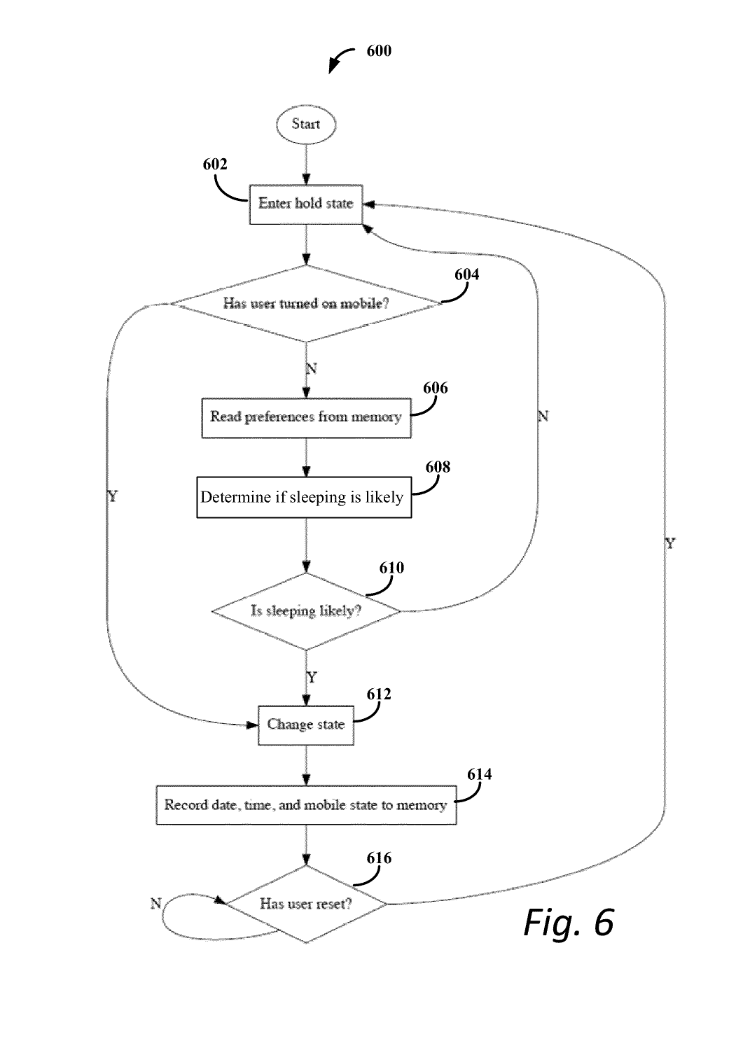

Referring now to the embodiments in more detail, FIG. 6 schematically illustrates a method of operating infant accessory system 100. More particularly, the figure shows a flow diagram depicting an exemplary process 600 of lulling or calming an infant with electrically-actuated outputs (208, 210, 212).

The methodology may, without limitation, incorporate a state machine that allows initiation of a lulling sequence based on stored user preferences. When the process 600 is first started, the system 100 optionally may enter a hold state 602. The system then determines (in state 604) whether the user has turned on the electrically-actuated outputs (referred to in the figure as a “mobile”).

The system 100 may then check (in state 606), in a memory address or buffer associated with an input of the user, to determine if a user has indicated a change of the output settings of the electrically-actuated outputs. The memory address or buffer may be linked, for example, to: a switch on the system 100 or a command to be received via the communication port from an external device (for example, the server 104, the remote device 106, or the communication device 108). To that end, the user may manually initiate the control state to initiate the electrically-actuated outputs (208, 210, 212) remotely via the network or directly via an input at the system 100. The system 100 then determines if the infant has fallen asleep (in state 608). This determination may be based on the rate of change of the infant's movement being below a specified threshold and is thus indicative that the infant is likely sleeping. If a data value at the memory address or the buffer indicates that sleeping is likely (in state 610), the system 100 then enters a change state (in state 612) to disable the output of the electrically-actuated outputs. The system 100 then stores (in state 616) information about the change to memory 226 before waiting (in state 616) for the user to reset. Of course, the system 100 may disable the output of the electrically-actuated outputs in a similar manner to the entry of the state to enable the activation of the electrically-actuated outputs.

The data value may change by a setting that the user provides to the system 100. For example, the setting may include program time and control levels, such as the specific output setting for the electrically-actuated outputs (208, 210, 212). The setting may also be rules or conditions for the system 100. To that end, the user may provide user customizable rules to initiate the controls. The customization rules, for example, may be associated with the use of the electrically-actuated outputs (208, 210, 212), observed environmental conditions associated with the infant, observed infant movements, and/or detected proximity of the user to the system 100.

If the data value indicates that the user does not want a change in output settings, the system 100 reads (in state 606) information associated with output settings from the memory 226 and then determines (in state 608) if the infant 202 is likely going to fall asleep in the near future. If the system 100 determines (in state 610) that a change of state is likely desired, the system 100 changes to an output state to enable the electrically-actuated outputs (208, 210, 212). The system 100 then records (in state 614) information about the change to memory 226 and waits (in state 616) for the user to reset the system 100, such as for the time the user puts the infant 202 in the infant bed 204.

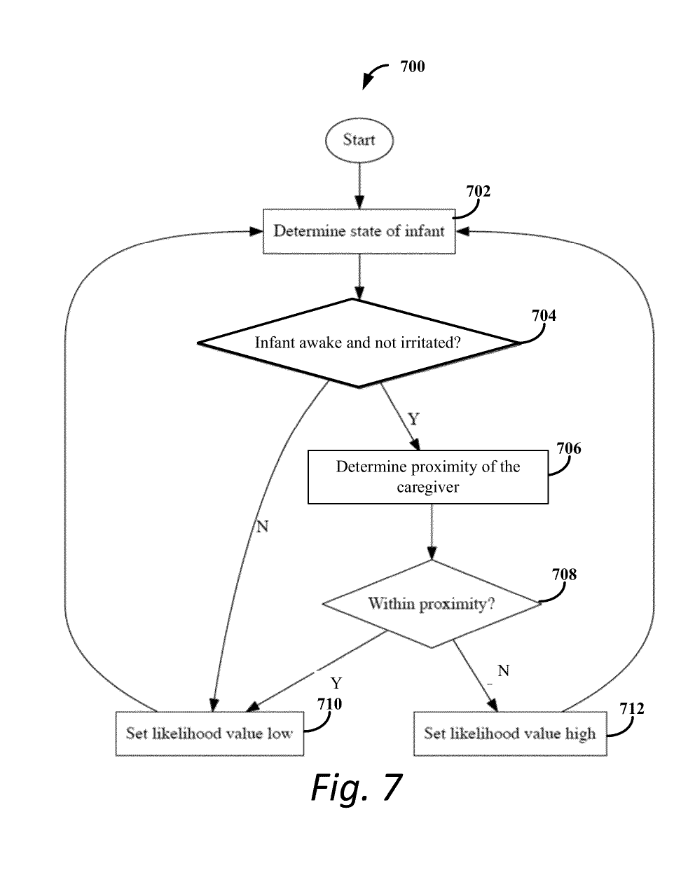

FIG. 7 schematically illustrates a method of determining a likelihood value for operating the infant-bed accessory system 100 based on the infant's state and the user's proximity according to an embodiment of the invention. The process 700 begins with the system 100 determining (in state 702) either a wake state or a sleep state of the infant. The wake/sleep state may, for example, be received from the infant monitor 102 or determined from collected information (for example, movement information associated with the infant) from the motion sensor 218. The system 100 then determines (in state 704) whether the infant is either awake or not asleep. Additionally, in state 704, the system 100 may determine a mood state of the infant. The mood state may include, but is not limited to, whether the infant is irritated, which may be based on a detection of: rapid breathing by the infant, elevated level of perspiration, elevated level of movements (for example, of the upper and lower extremities), crying, hunger, or a wet swaddle. Upon the system 100 determining that the infant is awake and not irritated, the system 100 may then determine the proximity of the user to the system 100 (state 706). The proximity may be determined based on spatial information (for example, GPS data or mobile location information) or wireless network information (for example, a presence of a wireless device or the signal strength of the wireless signal or the presence of a broadcast message). The system 100 may evaluate the spatial information to determine (in state 708) whether the user is within a specified distance from the system 100. The specified distance may be provided as an input from the user. If the user is not within proximity, the system may set (in state 712) the likelihood value that activation of the electrically-actuated outputs (208, 210, 212) is likely, as high. If the system 100 determines either the infant to be awake and irritated or the caregiver being within proximity, the system 100 may set (in state 710) the likelihood value as low.

To determine the mood state, the system 100 may combine data values associated with the state of the infant, the physiological responses of the infant, the movements of the infant, the environmental condition around the infant, and the orientation of the infant. In an embodiment, the combination may, for example, be based on a weighted sum of various sensor readings, such as those from a microphone, an accelerometer, a conductance sensor, an inductive sensor, a conductivity sensor, a light sensor, and a thermal sensor. In another embodiment, the combination may be based on statistical analysis of the various sensor readings associated with the infant and the user's location/proximity to the system 100 to determine the infant's mood. Such statistical analysis may include regression analysis, clustering analysis, and/or modeling analysis.

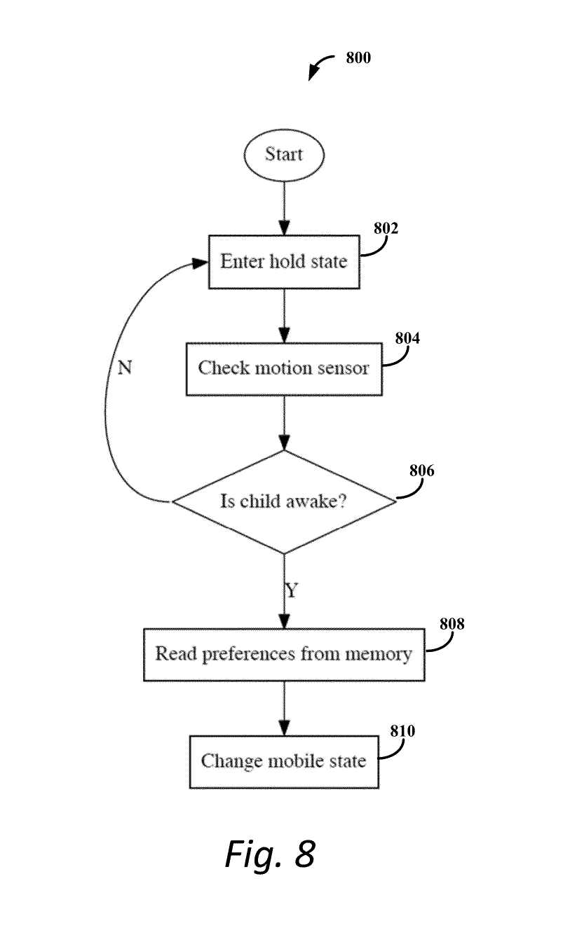

FIG. 8 schematically illustrates another method of controlling the output of the electrically-actuated outputs (208, 210, 212) according to another embodiment of the invention. The process 800 begins with the system 100 entering (in state 802) a hold state. The system then checks (in state 804) for the movements of the infant, such as from the motion sensor 218, and then determines (in state 806), a likelihood value of the infant being awake or asleep. The system 100 may then read (in state 806) preferences from memory 226 of either condition existing. As such, the caregiver may provide instructions to enable the electrically-actuated outputs (208, 210, 212) when the infant is awake or to disable the electrically-actuated outputs (208, 210, 212) when the infant is asleep. Based on the preferences, the system 100 changes (in state 810) the output of the electrically-actuated outputs. Additionally, the system 100 may record (in state 810) the time for the infant to fall asleep.

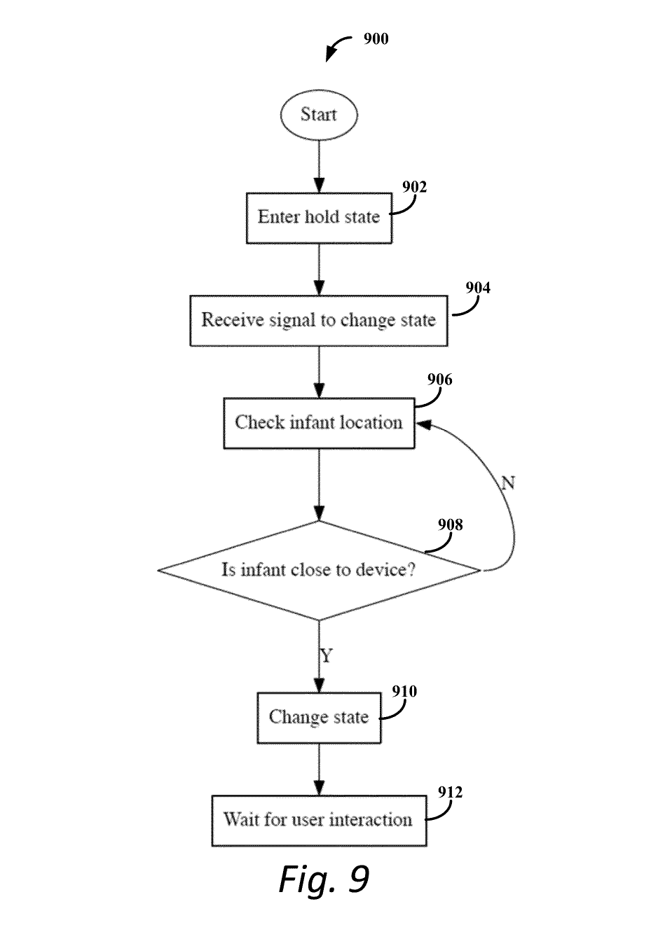

FIG. 9 schematically illustrates another method of controlling the system according to another embodiment of the invention. The system 100 is configured to be in the monitoring state only when the infant 202 is in the infant bed 204 or in proximity to the infant bed 204. The process 900 begins with the system 100 entering (in state 902) a hold state. The system then checks (in state 904) for received signals, such as from the communication port 228, to initiate the output of the electrically-actuated outputs (208, 210, 212). The process 900 then checks (in state 906) the infant's location and determines (in state 908) whether the infant is in the infant bed 204 or is in proximity thereto. The system 100 then changes (in state 910) the state to activate the electrically-actuated outputs if it determines the infant to be nearby and waits (in state 912) for further user interaction.

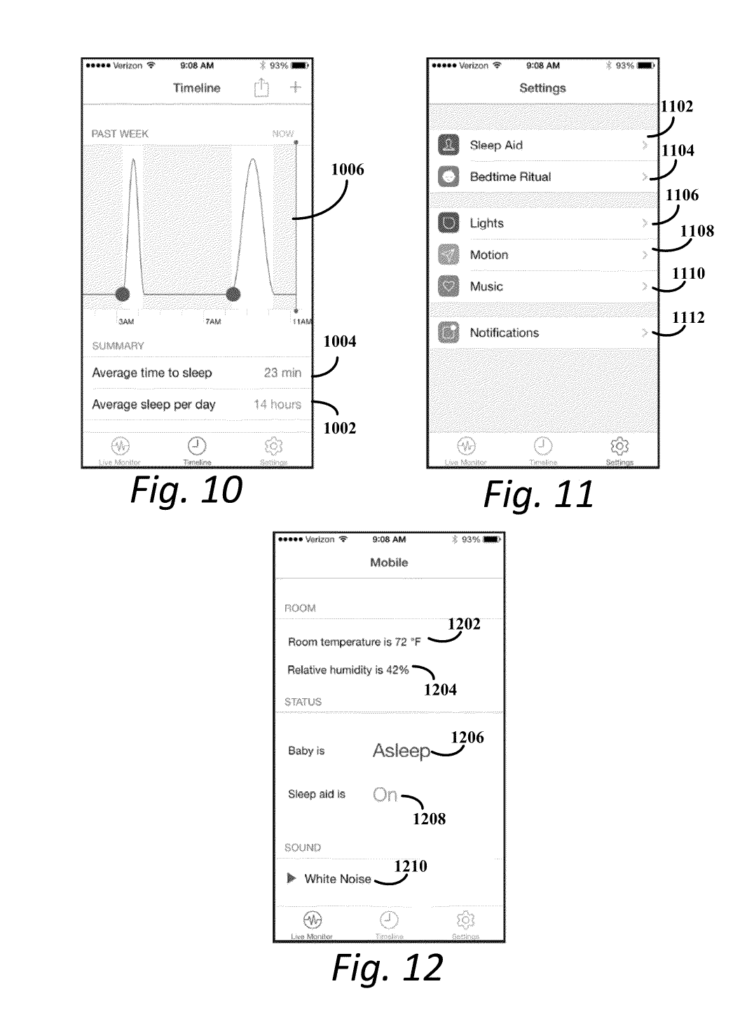

The system 100 may send alerts, notification and/or reports to the user. FIGS. 10-12 show exemplary display screens that may, for example, be displayed on a personal computing device in accordance with various embodiments of the invention.

FIG. 10 shows a live monitor mode associated with the infant sleeping, in which contemporaneous data 1006 from the sensors 218 may be displayed and/or various alerts. For example, and without limitation, current sleep, wake state, and mood state may be viewed. Summary sleep data over the past several weeks, including the average sleep time 1002 that the infant has per day, the average time 1004 for the infant to fall asleep after the electrically-actuated outputs (208, 210, 212) actuated, may be viewed. Other display modes, such as, without limitation, live monitor, timeline, settings, and mobile, may be viewed by selecting the appropriate button at the bottom of the screen.

FIG. 11 shows an alert setting mode, which allows the user the capability to select and enable various settings and alerts to the user (i.e., caregiver). The user may provide permission or preferences to the system 100 that she or he wishes to receive from the system in the notification setting 1112. For example, the user may indicate to the system 100 that the user wish to be notified, such as by a message, when the system 100 determines the infant is asleep or is likely asleep. The user may also wish to be notified, such as by a message, when the system 100 determines that the infant is awake. The user may also disable the notifications of the recommended time and recommended environment conditions to the user.

The caregiver may use the information to adjust settings and/or to help them in putting the infant to sleep. In particular, the messages and notification are configured to assist the caregiver in sleep training of the infant. The recommendations or alerts are focused on determining the ideal conditions to promote healthy sleep habits. To this end, the system 100 may provide a recommended time, a humidity setting, a temperature setting, and/or a recommended stimulus output of the system 100 (for example, lights, sounds, or motion). The recommendations may be configurable by the user. For example, the user may specify a recommendation based on, and without limitations, the time of day (for example, daytime and night time or a specific time of the day) and the day of the week (for example, weekend and weekend or a specific day).

Examples of messages provided by the system 100 include, but are not limited to: “If you put your child to bed now, the child will fall asleep faster”, or “If you put your child to bed fifteen minutes from now, your child will have a better night sleep.” The message may also be, “If you put your child to bed with the blue lights, she will likely fall asleep faster.” The message may be a simple message to assist in the setting of the electrically-actuated outputs (208, 210, 212), including “You should try this sequence.” Of course, any combinations of the message may be employed. The alerts or recommendations are preferably settings that may be configured by the user through a portal operating on the mobile phone or Internet website portal.

The settings may include, without limitation, a sleep aid setting 1102 and a bedtime ritual setting 1104. The sleep aid setting 1102 allows the user to enable the monitoring of the infant 202 and provides permission for the system 100 to output electrically-actuated outputs (208, 210, 212) when the infant is awake or likely to be awake. The bedtime ritual setting 1104 allows the user to provide a schedule to the system 100, in particular a time when to enable the electrically-actuated outputs (208, 210, 212).

The lights setting 1106, the motion setting 1108, and the music setting 1110 allow the user to specify the output settings for the electrically-actuated outputs (208, 210, 212). In the lights setting 1106, the user may, for example, specify a single color or a set of colors to be displayed, the brightness levels, and whether to pulse the light outputs. In the motion setting 1108, the user may specify whether to enable the motor 218. In the music setting 1110, the user may select a list of sound output to play.

FIG. 12 shows environmental information around the mobile, including the current room temperature 1202, the current humidity level 1204, the state of the infant 1206, the state of the electrically-actuated outputs (1208), and the current sound output of the system 100 (1210).

The embodiments of the invention described above are intended to be merely exemplary; numerous variations and modifications will be apparent to those skilled in the art. All such variations and modifications are intended to be within the scope of the present invention as defined in any appended claims.

For example, the system 100 may also be used to relay sound and images in proximity to the system 100 (in the nursery) to a user portal to allow the caregiver to directly and remotely monitor the infant. The system 100 may also relay sound from the user portal (or a telephone call) to allow the caregiver to talk to the infant remotely.

What I claim is:

1. A method of controlling an infant mobile, the method comprising:
connecting the mobile to a network;
transmitting information between a personal computing device and the infant mobile through the network; and
changing the state of the infant mobile based on the transmitted information.

2. The method according to claim 1, wherein the infant mobile includes at least one of, a motor to move a rotary member, a lamp, an LED, a projector, or a speaker.

3. The method according to claim 2, wherein the at least one lamp, LED, or projector is configurable in intensity, color, or pattern.

4. The method according to claim 1, wherein the network is a home wireless network, a cellular network, or a point-to-point connectivity with a personal computing device.

5. The method according to claim 1, wherein the infant mobile includes an electronic relay for powering an electrical outlet.

6. An infant-bed accessory comprising:
a sensory-stimulus member configured to be placed in proximity to the subject's bed, the sensory-stimulus member having at least one configurable electrically-actuated output selected from a group consisting of lights, sounds, and motion;
a controller operatively connected to the sensory-stimulus member to control the at least one configurable electrically-actuated output,
wherein the controller is configured to (i) receive signals associated with an awake state or a sleep state of the subject and to (ii) cause a sequence of the configurable electrically-actuated output that has a determined highest likelihood of lulling the subject to sleep based on the received signals.

7. The infant-bed accessory according to claim 6, wherein the controller determines the sequence by:
maintaining a record, in memory, of a plurality of values associated with a running-configuration of the configurable electrically-actuated output, the controller updating the plurality of values based on a time for the subject to fall asleep while the running-configuration is initiated.

8. The infant-bed accessory according to claim 6, wherein the controller includes a memory configured to store a control sequence for the plurality of lights, the sequence including at least one parameter selected from a group of parameters associated with a light's color, a light's intensity, and a light's pattern.

9. The infant-bed accessory according to claim 6, wherein the sensory-stimulus member includes an electronic speaker operatively connected to and controlled by the controller, and wherein the controller includes a memory for storing at least one audio sequence to output to the electronic speaker.

10. The infant-bed accessory of claim 9, wherein the controller is configured to receive an audio file over a communication network, the controller storing the received audio file in the memory as a stored audio sequence to output to the electronic speaker.

11. The infant-bed accessory according to claim 6, further comprising:
a switch member for gating power to an AC socket, the switch member being operatively connected to the controller.

12. The infant-bed accessory according to claim 6, wherein the sensor is a type selected from a group consisting of an infrared sensor, a capacitance sensor, a temperature sensor, a light sensor, a chemical sensor, motion sensor, a humidity sensor, a vibration sensor, a pressure sensor, an electric field sensor, a sound sensor, a stretch sensor, a camera, and a biosensor.

13. The infant-bed accessory according to claim 6, wherein the sensor is configured to sense at least one physical property of the subject selected from a group consisting of: the subject's heart beating rate, the subject's oxygen saturation level, the subject's movement, the subject's location, the subject's noise level, the subject's body temperature, the subject's skin conductivity; the subject's skin moisture level, and the subject's infrared light emission.

14. The infant-bed accessory according to claim 6, wherein the sensory-stimulus member is configured to rotate when initiated.

15. The infant-bed accessory according to claim 6, wherein the sensory-stimulus member is configured to emit sound when initiated.

16. The infant-bed accessory according to claim 6, wherein the sensory-stimulus member is configured to emit light when initiated.

17. The infant bed accessory according to claim 6, wherein the information transmitted includes the location of a personal computing device.
